(19) United States
(12) Patent Application Publication
(10) Pub. No.: US 2011/0045094 A1
(43) Pub. Date: Feb. 24, 2011

(54) ENCAPSULATED QUANTUM DOT

(75) Inventors: Ming Yong Han, Singapore (SG); Yin Win Khin, Singapore (SG); Enyi Ye, Singapore (SG); Lai Ying Ng, Singapore (SG)

Correspondence Address: Joseph R. Baker, APC;Gavrilovich, Dodd & Lindsey LLP, 4660 La Jolla Village Drive, Suite 750, San Diego, CA, 92122, US

(73) Assignee: AGENCY FOR SCIENCE, TECHNOLOGY AND RESEARCH, Singapore, SG (SG)

(21) Appl. No.: 12/593,877

(22) PCT Filed: Mar. 28, 2008
(86) PCT No.: PCT/SG2008/000097

§ 371 Date: Sep. 22, 2010

Related U.S. Application Data
(60) Provisional application No. 60/921,045, filed on Mar. 30, 2007.

(51) Int. Cl. A61K 9/14; C09K 11/02; A61P 35/00; B82Y 5/00; B82Y 15/00

(52) U.S. Cl. 424/497; 252/301.36; 977/774; 977/906

(57) ABSTRACT

A particle comprising a quantum dot encapsulated by an amphiphilic polymer. These particles are suitable for use in biological and biomedical research and may emit fluorescence and may be water-soluble and biocompatible. The encapsulated quantum dot may be introduced into a living system without any substantial toxic or immunological effects.

### TECHNICAL FIELD

The present invention generally relates to an encapsulated quantum dot.

### BACKGROUND

Fluorescence techniques are widely used in biological and biomedical research and thus there is an increasing demand for the development of more advanced fluorescent probes. Organic fluorophores have been used for fluorescent tagging of cells and biomolecules. Unfortunately, their use is restricted by their narrow excitation spectra, broad emission spectra and weak photostability. Inorganic semiconductor quantum dots (QDs) have been proposed as promising alternatives for fluorescence labeling in multicolor biological imaging and detection applications. QDs possess a composition-dependent, shape-dependent and size-dependent luminescence with absorption and emission bands that scale with the bulk band gap energy of the material and the final diameter of the QD clusters.

Highly luminescent QDs have relatively long fluorescence lifetimes and are useful in tagging biomolecules for ultrasensitive biological detection and medical diagnostic applications. Compared to conventional organic fluorophores, QDs have strong, narrow, and symmetric fluorescence emission and are photochemically stable with a quantum yield (the ratio of emitted to absorbed photons) as high as 90%. Their low photodegradation rates make continuous or long-term real-time monitoring of slow biological processes or tracking of intracellular processes that are not possible with conventional organic fluorophores. Therefore, QDs have potential to replace organic fluorophores as fluorescence probes for cell labeling studies. Since QDs are inorganic solids, they can be expected to be more robust than organic fluorophores (e.g. towards photobleaching) and in addition, they can also be observed with high resolution by electron microscopy.

Thus, luminescent QDs are desirable fluorophores for biological imaging, as their fluorescence emission wavelength can be continuously tuned from the near ultraviolet, throughout the visible and into the near-infrared spectrum, thereby spanning a broad wavelength range of 400-1350 nm.

QDs of varying particle size will exhibit different wavelength absorbance. Accordingly, by using a number of QDs having different particle sizes, a single wavelength can be used for simultaneous excitation to detect different optical activities.

Although development of QDs for biological labeling opens up new possibilities for multicolor detection and diagnostics, QDs themselves are not water soluble, not biocompatible and chemically stable, and do not have functional groups for covalent conjugation with biomolecules. Given these properties, the utility of QDs for biological application is currently limited. High quality QDs (in terms of crystallinity and size distribution) are synthesized in nonpolar solvents with hydrophobic coatings such as trioctyl phosphine oxide (TOPO). However hydrophobic coatings are not suitable for use in vivo.

Efforts have been made to surface modify single QDs to solve the above problems and to allow the successful use of QDs as biocompatible fluorescent probes or bio-markers. However, surface modification of QDs is very dependent on the surface chemistry of QDs. The surface of the QDs may be tailored to interact with the biological samples either through electrostatic and hydrogen-bonding interactions or through ligand-receptor interaction, such as for example, avidin-biotin interaction.

There have been some studies undertaken on the surface modification of QDs, such as conjugation of mercaptoacetic acid (MAA) and coating of silica on ZnS-capped or -uncapped QDs, which have proved promising. However, a disadvantage of QDs capped with small molecules, such as MAA, is that they are easily degraded by hydrolysis or oxidation of the capping ligand. Silica coating can be used to coat QDs or encapsulate QDs to form silica nanospheres. However, a disadvantage of silica coating requires the surface of QDs to be modified first with special silane surfactants.

For biological applications, single QDs are currently surface modified by substituting these hydrophobic coating molecules with various hydrophilic capping agents of bifunctional linkers. The use of capping agents allows QDs to solubilize in an aqueous medium and provide functional groups which can be conjugated to biomolecules for particular applications. However, this is a complicated process and also requires the use of non-biocompatible organic ligands. Therefore, the inflexibility of the capping agent limits the use of the resultant QD as fluorophore probes. The multi-valency of currently available QD bioconjugates further precludes their use for labeling only a single molecule in live cells. The fact that their configurations cannot accommodate drug loading is a major impediment to make them multi-functional nanostructured devices in biomedical applications.

There is therefore a need for a simpler and more feasible method to synthesise water-soluble and biocompatible QDs.

### SUMMARY

According to a first aspect, there is provided a particle comprising a quantum dot encapsulated by an amphiphilic polymer.

In one embodiment, the amphiphilic polymer substantially encapsulates the quantum dot, which is typically hydrophobic in nature. Advantageously, by encapsulating the quantum dot, the amphiphilic polymer may aid in allowing the quantum dot to exist in an aqueous medium while retaining its optical properties. Furthermore, by choosing an amphiphilic polymer that is biocompatible, the resultant encapsulated quantum dot may be introduced into a living system without any substantial toxic or immunological effects on the living system. The biocompatibility of the amphiphilic polymer may aid in the uptake of the encapsulated quantum dots into a cell of the living system.

In one embodiment, the disclosed particle is in the nanometer range.

In one embodiment, there is provided a fluorescent probe comprising a quantum dot encapsulated by an amphiphilic polymer, wherein the quantum dot is capable of exhibiting fluorescence.

According to a second aspect, there is provided the use of a particle comprising a quantum dot encapsulated by an amphiphilic polymer as a fluorescent probe.

Advantageously, this may allow the disclosed particles to be used as delivery vectors of quantum dots and can be effectively taken up by cells (as observed by clear in vivo fluorescence imaging). Even more advantageously, as a result of efficient cell uptake, the disclosed particles may also serve as a model system for the study of cell-uptake behaviors of the polymeric particles, which can be used to screen the existing polymer candidates for non-fluorescent expensive drug delivery and controlled release. Even more advantageously, the disclosed particles may be used in various bio-imaging techniques to study their biodistribution and intracellular pathway, to track the mechanism and efficacy of drug delivery device in cellular level and also to evaluate the polymers used to develop efficient drug delivery devices.

According to a third aspect, there is provided use of a particle comprising a quantum dot and a therapeutic agent encapsulated by an amphiphilic polymer for controlled release of said therapeutic agent in a patient, wherein said quantum dot is capable of being optically detected in vivo during said release.

Advantageously, the optical property of the quantum dot may aid a medical practitioner in determining the efficacy and metabolic pathway of the therapeutic agent as it is administered to a mammal and absorbed by mammal.

According to a fourth aspect, there is provided a method of preparing an encapsulated quantum dot comprising the step of introducing an aqueous solvent to a quantum dot in mixture with an amphiphilic polymer dissolved in an organic solvent to thereby precipitate said polymer and encapsulate said quantum dot.

Also disclosed is the use of a particle comprising a quantum dot and a therapeutic agent encapsulated by an amphiphilic polymer in the manufacture of a medicament for treating a patient, wherein said quantum dot is capable of being optically detected in vivo in said patient during said release of said therapeutic agent. The patient may be suffering from cancer and the therapeutic agent may be an anti-cancer drug.

### DEFINITIONS

The following words and terms used herein shall have the meaning indicated:

The term “quantum dot” is to be interpreted broadly to include any semiconductive or metallic nanoparticle that is capable of emitting a light signal. The particle size of the nanoparticle is typically about 1 nm to about 1000 nm, more typically less than about 2 nm to about 10 nm. The shape of the quantum dot is not limited and may be in the shape of a sphere, a rod, a wire, a pyramid, a cube, or other geometric or non-geometric shapes. The colour of the light emitted by the quantum dot depends on a number of factors that include the size and shape of the quantum dot. For example, a quantum dot with a larger particle size emits light with a lower energy as compared to a quantum dot made of the same material but with a smaller particle size.

The term “amphiphilic polymer” is to be interpreted broadly to include any polymer that has a hydrophobic part and a hydrophilic part. The amphiphilic polymer may have hydrophilic side chains grafted on or attached to a hydrophobic polymer backbone or the amphiphilic polymer may have hydrophobic side chains grafted on to a hydrophilic polymer backbone. The amphiphilic polymer may be a copolymer of two or more types of monomers, each monomer having a different degree of hydrophobicity or hydrophilicity. In embodiments where the amphiphilic polymer is a copolymer, at least one of the monomers is a hydrophobic monomer and at least one of the other monomers is a hydrophilic monomer.

The term “hydrophobic” is to be interpreted broadly to refer to a substance, such as a monomer or part thereof or a polymer or part thereof or a quantum dot, that exhibit a low intermolecular attraction for aqueous solvents such as water. Alternatively, the term “hydrophilic” is to be interpreted broadly to refer to a substance, such as a monomer or part thereof, or a polymer or part thereof, that exhibits a high intermolecular attraction for aqueous solvents such as water.

The amphiphilic polymers disclosed herein may be biocompatible and may be biodegradable and/or bioresorbable.

The term “biocompatible” is to be interpreted broadly to denote a polymer that is compatible with living tissue or a living organism by not being toxic or injurious and by not causing immunological reaction to that tissue or living organism.

The term “biodegradable” is to be interpreted broadly to refer to a polymer that breaks down into oligomeric and/or monomeric units over a period of time, typically hours to months, when implanted or injected into the body of a mammal.

The term “bioresorbable” is to be interpreted broadly to refer to a polymer whose degradative products are metabolized in vivo or excreted from the body via natural pathways.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/− of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DISCLOSURE OF OPTIONAL EMBODIMENTS

Exemplary, non-limiting embodiments of a particle comprising a quantum dot encapsulated by an amphiphilic polymer will now be disclosed.

The particle may be of a size in the nanometer range.

The particle may be substantially spherical in shape. In one embodiment, the diameter of the substantially spherical particle may be in the range selected from the group consisting of about 50 nm to about 500 nm; about 50 nm to about 400 nm; about 50 nm to about 300 nm; about 50 nm to about 200 nm; about 50 nm to about 100 nm; about 100 nm to about 500 nm; about 100 nm to about 200 nm; about 100 nm to about 300 nm and about 100 nm to about 400 nm. Advantageously, the disclosed nanoparticles are about 100 to about 300 nm in size and are therefore suitable for use as carriers to incorporate drug for drug delivery and as means for controlled drug release.

The quantum dot may be substantially hydrophobic. The quantum dot may be made from at least one element selected from Group IIB, Group IVA, Group VA, Group IIIA, Group IIA or Group VIA of the Periodic Table of Elements. The quantum dot may be made of a material such as, but not limited to, CdO, CdS, CdSe, CdTe, CdSeTe, CdHgTe, ZnS, ZnSe, ZnTe, ZnO, MgTe, MgS, MgSe, MgO, GaAs, GaP, GaSb, GaN, HgO, HgS, HgSe, HgTe, CaS, CaSe, CaTe, CaO, SrS, SrSe, SrTe, SrO, BaS, BaSe, BaTe, BaO, InAs, InP, InSb, InN, AlAs, AlN, AlP, AlSb, AlS, PbO, PbS, PbSe, PdTe, Ge, Si, ZnO, ZnS, ZnSe, ZnTe and combinations thereof.

The quantum dot may be of a core-shell structure. Exemplary shell material include, but are not limited to, ZnO, ZnS, ZnSe, ZnTe, CdO, CdS, CdSe, CdTe, MgS, MgSe, GaAs, GaN, GaP, GaAs, GaSb, HgO, HgS, HgSe, HgTe, InAs, InN, InP, InSb, AlAs, AlN, AlP, AlSb, or combinations thereof, optionally with the inner shell comprising at least one element selected from Group IIB, Group IVA, Group VA, Group IIIA, Group IIA or Group VIA of the Periodic Table of Elements.

In one embodiment, the quantum dot has an inner core of CdSe and a outer shell of ZnS.

The amphiphilic polymer may be biocompatible. The amphiphilic polymer may not have any toxic or immunological effects on a living system. The amphiphilic polymer may be substantially tolerated by cells or organs of a living system.

The biocompatible amphiphilic polymer may be selected from the group consisting of polyesters, poly(orthoester)s, polyanhydrides, poly(aminoacid)s, poly(pseudo amino acid)s, and polyphosphazenes.

In one embodiment, the biocompatible polymer may be a polyester selected from the group consisting of poly(lactic acid)s, poly(glycolic acid)s, copolymers of lactic and glycolic acid, copolymers of lactic and glycolic acid with poly(ethylene glycol), poly(ε-caprolactone)s, poly(3-hydroxybutyrate)s, polybutyrolactones, polypropiolactones, poly(p-dioxanone)s, poly(valerolactone)s, poly(hydrovalerate)s, poly(propylene fumarate)s and derivatives thereof.

The polyester that is a copolymer of lactic and glycolic acid may be selected from the group consisting of (D-lactic-co-glycolic acid), poly(L-lactic-co-glycolic acid) and poly(D,L-lactic-co-glycolic acid). In one embodiment, the ratio of lactic acid and glycolic acid in the copolymer may range from about 1:10 to about 10:1.

In another embodiment, the biocompatible polymer may be a hydroxyl or carboxyl end-functionalized linear, dendritic or star-shaped polyester.

The biocompatible polymer may be a polyester having a molecular weight of about 1,000 Da to about 100,000 Da.

In one embodiment, the biocompatible polyester is poly(D,L-lactic-co-glycolic acid) (PLGA).

The biocompatible amphiphilic polymer may have a hydrophobic inner core surrounded by a hydrophilic outer skin.

The hydrophilic outer skin of the biocompatible amphiphilic polymer may comprise hydrophilic functional groups. The hydrophilic functional groups may be selected from the group consisting of hydroxyl groups, carboxyl groups, ether groups, sulfide groups, ester groups, ethoxy groups, phosphonyl groups, phosphinyl groups, sulfonyl groups, sulfinyl groups, sulfonic acid groups, sulfinic acid groups, phosphoric acid groups, phosphorous acid groups, amino groups, amide groups, quaternary ammonium groups, and quaternary phosphonium groups.

The inner core of the biocompatible amphiphilic polymer may comprise hydrophobic functional groups. The hydrophobic functional groups may be selected from the group consisting of linear or branched alkyl groups, aryl groups, alkenyl groups, alkynyl groups, alkylacrylamide groups, substituted or unsubstituted alkylacrylate groups, and alkylaryl groups.

The amphiphilic polymer may be a polyester polycation copolymer. In one embodiment, the polyester polycation copolymer may be a diblock copolymer comprising a hydrophobic polyester block bonded to a hydrophilic polycation. In another embodiment, the polyester polycation copolymer may be a graft copolymer comprising a hydrophobic polyester portion and a hydrophilic cation portion.

The polycation may be selected from the group consisting of poly(L-serine ester), poly(D-serine ester), poly(L-lysine), poly(D-lysine), polyornithine, and polyarginine. In one embodiment, the polycation may have a molecular weight of about 500 to about 10,000.

The disclosed particle may further comprise a therapeutic agent encapsulated by the amphiphilic polymer. In one embodiment, the amphiphilic polymer may encapsulate a mixture of the therapeutic agent and quantum dot therein.

The therapeutic agent may comprise an anticancer agent such as, but not limited to, dideoxyinosine, camptothecin, floxuridine, 6-mercaptopurine, doxorubicin, daunorubicin, I-darubicin, cisplatin, methotrexate, carboplatin, oxaliplatin, mechlorethamine, cyclophosphamide, chlorambucil, vinca alkaloid, taxane, vincristine, vinblastine, vinorelbine, vindesine, etoposide or teniposide.

It is to be appreciated that the type of therapeutic agent used is not particularly limited to those mentioned above but includes any therapeutic agent that is suitable to be in an admixture or to be coupled to a quantum dot.

The disclosed particle may be used as an optical marker in vivo. This may allow a medical practitioner to trace the route of the particle when it is administered or injected into a mammal by detecting the light emitted by the quantum dot.

The disclosed particle may comprise a mixture of a therapeutic agent and a quantum dot encapsulated by the amphiphilic polymer. The light emitted by the quantum dot may aid in determining the metabolic pathway or organs targeted by the therapeutic agent when the particle is administered to a mammal.

The colour of the light emitted by the quantum dot may be linked to the presence of the therapeutic agent. For example, the therapeutic agent may be coupled to the quantum dot and hence increases the effective size of the quantum dot. As mentioned above, the size of the quantum dot is one of the factors that affects the colour of the light emitted from the quantum dot. Therefore, a larger particle sized quantum dot may emit a light that is of a different colour as compared to a smaller particle sized quantum dot. When the particle is administered to a mammal, the colour emitted by the quantum dot coupled with the therapeutic agent may change as the therapeutic agent is absorbed or taken up by the cells of the body, leading to a reduction in the effective size of the quantum dot. By determining the change in the colour emitted by the quantum dot as time passes, the efficacy and pharmacokinetics of the therapeutic agent in the body may be determined. This may be of use in imaging-guided chemotherapy, where quantum dots can be used as optical reporters. The pathway of the disclosed particle in vivo may be determined and controlled release of the therapeutic agent may occur at a desired position.

The disclosed particle may comprise a mixture of a quantum dot and a therapeutic agent encapsulated by the amphiphilic polymer. Advantageously, the disclosed particle may be used as a drug delivery vehicle for administration of the therapeutic agent into a mammal. The biodegradation of the amphiphilic polymer in the mammal body may aid in the release of the therapeutic agent at specific times, leading to a controlled release of therapeutic agent. As the therapeutic agent is released into the body, the quantum dot may aid in the optical detection of the therapeutic agent in vivo during the release of the therapeutic agent.

The disclosed particle comprising a mixture of a quantum dot and a therapeutic agent encapsulated by an amphiphilic polymer may be used to determine the inhibitory effects or therapeutic actions of the therapeutic agent on a foreign microorganism in the living system. The foreign microorganism may be a bacterium, a fungi or a virus that is causing a disease in a mammal. The therapeutic agent may react with the foreign microorganism and may be taken into the foreign microorganism. By observing the colour change in the quantum dot particle over a period of time, the therapeutic action of the therapeutic agent on the foreign microorganism can be determined as the mammal recovers from the disease.

The disclosed particle may be made from a method comprising the step of introducing an aqueous solvent to a quantum dot in mixture with an amphiphilic polymer dissolved in an organic solvent to thereby precipitate the polymer and encapsulate the quantum dot.

The method may comprise the step of mixing the aqueous solvent with the quantum dot in mixture with the amphiphilic polymer dissolved in an organic solvent, to thereby form a two-phase system made up of an organic phase and an aqueous phase. Mixing of the aqueous solvent and organic solvent to generate the two-phase system may be carried out by sonicating the aqueous-organic mixture for about 1 minute to about 5 minutes. In one embodiment, the time required for the sonicating step may be from about 1 minute to about 2 minutes.

As the amphiphilic polymer encapsulates the quantum dot, which is typically hydrophobic in nature, the hydrophilic tails of the polymer preferentially move away from the quantum dot while the hydrophobic tails of the polymer preferentially move toward the quantum dot. The addition of the aqueous solvent to the organic solvent may cause the amphiphilic polymer liquid to precipitate. During precipitation, the hydrophilic tails of the polymer that are further away from the quantum dot are attracted to the aqueous solvent, thereby encapsulating the quantum dot. Accordingly, the particle comprises a core shell of quantum dot with an outer skin consisting of an inner hydrophobic polymer portion adjacent the quantum dot and an outer hydrophilic polymer portion adjacent the inner hydrophobic polymer portion. The hydrophilic nature of the exposed polymer tails may aid in the solubilization of the encapsulated quantum dots in an aqueous solution. The aqueous solution is typically water, which is an easily obtainable and cost effective solvent.

The method may comprise the step of extracting the encapsulated quantum dots from the liquid mixture. Extraction and collection of the encapsulated quantum dots from the above two-phase system may be carried out by evaporating the organic phase and collecting the encapsulated quantum dots from the aqueous phase. The encapsulated quantum dots may be collected form the aqueous phase by further evaporation of the aqueous phase, centrifugation or filtration.

The collected encapsulated quantum dots may be washed with deionised water via centrifugation to substantially remove impurities. The organic solvent may be a halogenated solvent or an ether. The halogenated solvent may be a chlorinated solvent selected from the group consisting of dichloromethane, 1,2-dichloroethane, chloroform and 1,1,1-trichloroethane.

The aqueous solvent may be a polar compound such as water, alcohol, polyvinyl alcohol and mixtures thereof.

### BRIEF DESCRIPTION OF DRAWINGS

The accompanying drawings illustrate a disclosed embodiment and serve to explain the principles of the disclosed embodiment. It is to be understood, however, that the drawings are designed for purposes of illustration only, and not as a definition of the limits of the invention.

FIG. 1(a) shows a microscopy image of the quantum dot nanoparticles (QD-nanoparticles) at 10,000× magnification.

FIG. 1(b) shows QD-nanoparticles dissolved in water.

FIG. 1(c) shows QD-nanoparticles dissolved in water while being illuminated by an ultra violet (UV) lamp.

FIG. 1(d) shows a fluorescent microscopic image of the QD-nanoparticles.

FIG. 2(a) shows a confocal fluorescence image of the QD-nanoparticles uptake in CCD-112CoN cell lines after incubation with QD-nanoparticles.

FIG. 2(b) shows a confocal fluorescence image of the distribution of QD-nanoparticles in the cells.

FIG. 2(c) shows a confocal fluorescence image of stained nuclei showing individual cells.

FIG. 3(a) shows a confocal fluorescence image in CCD-112CoN cell lines after incubation with a mixture of QD-nanoparticles and DOX-nanoparticles.

FIG. 3(b) shows a confocal fluorescence image of stained nuclei showing individual cells.

FIG. 3(c) shows the distribution of QD-nanoparticles in the cells.

FIG. 3(d) shows the distribution of DOX-nanoparticles in the cells.

FIG. 4(a) shows a confocal fluorescence image of a single cell (taken from the CCD-112CoN cell lines) after incubation with a mixture of QD-nanoparticles and DOX-nanoparticles.

FIG. 4(b) shows the distribution of QD-nanoparticles in a single cell.

FIG. 4(c) shows distribution of DOX-nanoparticles in a single cell.

FIG. 5(a) shows a scanning electron microscope (SEM) image illustrating the degradation of DOX-nanoparticles.

FIG. 5(b) is a graph showing a DOX release profile representing the percent of cumulative DOX released from DOX-nanoparticles.

FIG. 6 shows a confocal fluorescence image of the QD-NPs uptake in NCI-H1299 cell lines after incubation with QD-nanoparticles.

FIG. 7 shows a schematic diagram of a plurality of quantum dots encapsulated in PLGA.

FIG. 8 shows the cellular uptake of quantum dots encapsulated with polymer via endocytosis and invagination of the cell membrane.

FIG. 9 shows a simplified process flow chart of the modified emulsification solvent evaporation method for encapsulating QDs in PLGA.

### DETAILED DESCRIPTION OF DRAWINGS

Referring to FIG. 7, there is shown a typical quantum dot (QD) 16 having a core-shell structure, comprising of a cadmium selenium (CdSe) core 26 covered by a zinc sulphide (ZnS) outer shell 24. The ZnS shell is conjugated with aliphatic hydrocarbon chains 22 which are hydrophobic in nature. The hydrophobic aliphatic hydrocarbon chains 22 on the ZnS shell 24 of the quantum dot 16 render it insoluble in aqueous solvents. However, after being encapsulated by an amphiphilic polymer, such as poly(lactic-co-glycolic acid) (PLGA) 20, the aliphatic hydrocarbon chains 22 of the QD 16 interact with the hydrophobic functional groups of the PLGA 20 to form a QD-loaded polymer particle 28. The QD 16 is substantially locked within the polymer's 20 hydrophobic inner core. Advantageously, the hydrophilic external surface of the polymer 20 serves to facilitate transportation of the QD-loaded polymer particle 28 in the systemic circulation of the human body due to its increased solubility.

FIG. 8 shows the proposed mechanism of cellular uptake of the QD-loaded polymer particle 28. The QD-loaded polymer particle 28 is unable to enter the bi-layered plasma membrane 10 of a typical cell due to its hydrophilic (polar) functional groups on the surface of the polymer PLGA 20. Accordingly, in order to bypass the hydrophobic plasma membrane 10, the QD-loaded polymer particle 28 must be transported into the cell via the process of endocytosis. Hydrophillic interactions between the polymer PLGA 20 and the plasma membrane 10 causes the plasma membrane 10 to fold inwards and surround the QD-loaded polymer particle 28. The plasma membrane 10 eventually envelops the QD-loaded polymer particle 28 completely, thereby forming a vesicle 14. The QD-loaded polymer particle 28 is thus transported into the cytoplasm 12 of the cell through the invagination 18 of the plasma membrane 10. Furthermore, transportation of particles to cells and accumulation in cells may be due to phagocytosis, pinocytosis, and/or cytoskeletal, organelle and other particle transport mechanisms.

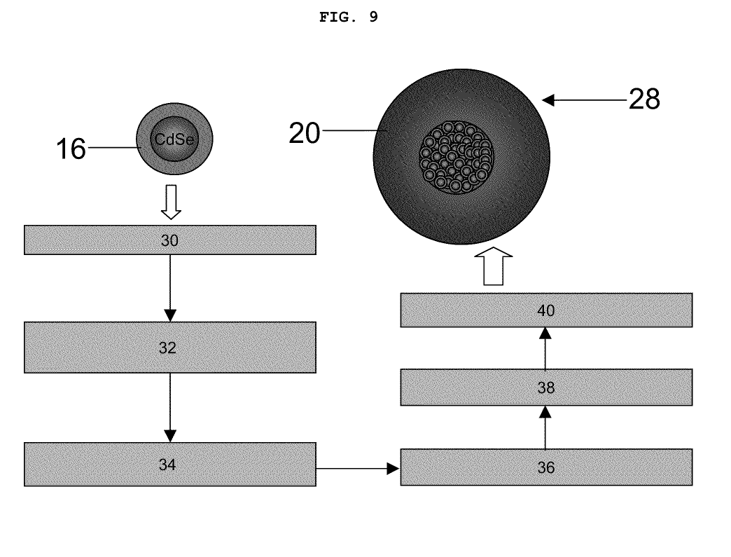

FIG. 9 is a schematic diagram showing a simplified process of the emulsification solvent evaporation method for encapsulating QDs 16 in the polymer PLGA 20 to form the QD-loaded polymer particle 28.

In the first step 30, the purified quantum dots 16, polymer PLGA 20 and dichloromethane (DCM) are mixed together to form a suspension of the quantum dots 16 in an organic solution.

A precipitation step 32 is then undertaken by introducing an aqueous solution of poly(vinyl alcohol) (PVA) in deionised water to the organic solution. The aqueous solution causes the PLGA coating the QD to solidify and thereby form the particle 28.

Sonication 34 is then carried out for about 1.5 minutes to further homogenize the mixture and thereby form an emulsion of the organic and aqueous solutions. Thereafter, extraction 36 of the resultant QD-loaded polymer particles 28 is carried out by evaporating the organic solvents from the emulsion. Evaporation is accomplished by magnetic stirring of the emulsion for 4 hours.

A washing step 38 is then undertaken with deionised water to further remove remaining organic solvent that may be in contact with the particles 28. Finally the polymer particles 28 are lyophilized at step 40 via freeze-drying.

### EXAMPLES

Non-limiting examples of the invention will be further described in greater detail by reference to specific Examples, which should not be construed as in any way limiting the scope of the invention.

### Example 1

Quantum dots encapsulated PLGA particles were prepared in the laboratory using a modified emulsification solvent evaporation method. Purified quantum dots having a core-shell structure, with CdSe as the core nanomaterial and ZnS as the shell material were first provided. 40 milligrams of poly(lactic-co-glycolic acid) (PLGA) from Sigma-Aldrich of St. Louis of Missouri of the United States of America was mixed with 2 ml of dichloromethane (DCM) to prepare a PLGA/DCM solvent. About 10 to about 15 milligrams of the purified QDs were then subsequently dissolved in 2 ml of the PLGA/DCM solvent to form an organic phase. About 24 ml of 2% w/v polyvinyl alcohol (PVA) dissolved in deionised water was used as an aqueous phase. Approximately 2 ml of the organic phase was then mixed with about 24 ml of the aqueous phase and the mixture was subsequently sonicated for about 90 s to form an oil-in-water emulsion. Evaporation was then carried out by placing the emulsion under magnetic stirring for about 4 hrs to remove the organic solvent. Thereafter, the particles were collected and washed with deionised water for at least 3 times via centrifugation. Finally, the washed particles were lyophilized by freeze-drying.

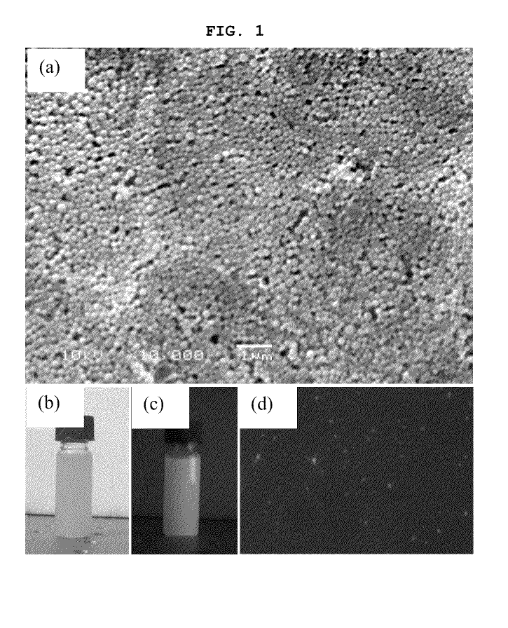

FIG. 1(a) shows a scanning electron microscopy (SEM) image depicting PLGA polymer encapsulating quantum dots (henceforth referred to as QD-nanoparticles) formed from the disclosed method at 10,000× magnification. The nanoparticles thus formed can be seen as discrete, substantially spherical particles with an approximate diameter of about 100 nm to 300 nm. FIG. 1(b) further shows a photograph of QD-nanoparticles dissolved in water. FIG. 1(c) shows an image of QD-nanoparticles in water, illuminated by a UV lamp. FIG. 1(d) shows a fluorescent microscopic image of QD-nanoparticles showing that the particles do in fact exhibit fluorescence. FIG. 1(b) shows that the QD-nanoparticles can be homogeneously dispersed in water, and brightly fluorescent as in FIG. 1(c) and FIG. 1(d). The encapsulation of QDs in the PLGA polymer to form nanoparticles not only renders the QDs the water dispersibility required for biological applications but also maintains their optical properties such that their optical properties are substantially comparable to those of QDs that have not been encapsulated by an amphiphilic polymer.

The nano-particles made in this example were used in the following examples.

### Example 2

Human colon fibroblast cells, CCD-112 CoN(CRL-1541, ATCC) were maintained in Dulbecco's Modified Eagle's Medium (DMEM) from Sigma-Aldrich of St. Louis of Missouri of the United States of America supplemented with 10% fetal bovine serum (FBS), 1.0 mM sodium pyruvate, 0.1 mM non-essential amino acids and 1% penicillin-streptomycin solution, and culture medium was replenished every day. To study cell uptake of nanoparticles, cells were seeded at 2.0×10⁴ cells/cm² in Lab-Tek chambered cover glasses and cultured as a monolayer at 37° C. in a humidified atmosphere containing 5% CO₂. The cell uptake of the nano-particles was initiated when the culture medium was replaced by the nano-particles suspension (500 μg/mL in culture medium) and the monolayer was further incubated for 2 hours at 37° C. At the end of experiment, the cell monolayer was washed 3 times with fresh pre-warmed phosphate-buffered saline (PBS) buffer to remove excess nanoparticles which were not associated to the cells. Cells were then fixed with 70% ethanol. Nucleus staining was carried out using propidium iodide (PI) or 4′-6-Diamidino-2-phenylindole (DAPI) to facilitate determination of the location of the nanoparticles in the cells. The samples were then mounted in a fluorescent mounting medium (Dako). Confocal fluorescent microscopy was performed using an Olympus FV500 system supported with a 60× water-immersion objective. Images were captured in section of 1024×1024 pixels without zoom and with a 0.0-5.0 μm z-step, and processed by FV10-ASW 1.3 Viewer.

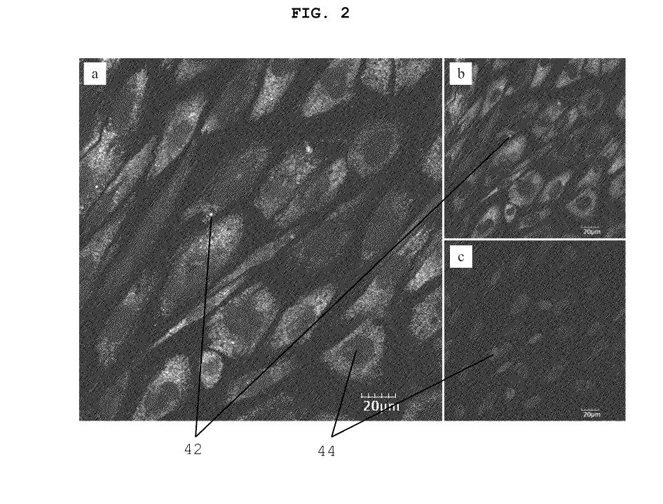

Referring now to FIG. 2, the confocal microscopy image shows the QD-nanoparticles 42 uptake in human colon fibroblast cells (CCD-112CoN) after a two hour incubation with QD-nanoparticles 42 at 37° C. followed by counterstaining of nucleus 44 by propidium iodide (PI). FIG. 2(a) shows dual-labeled cells visualized by overlaying images; FIG. 2(b) shows the QD-nanoparticles 42 distribution in cells; and FIG. 2(c) shows the image of the various cell nuclei 44 to facilitate distinguishing between the separate cells. The above results show excellent cell uptake of the QD-nanoparticles 42, suggesting that the hydrophilic surface of the nanoparticles did not impede its cellular transport. Furthermore, the substantial cell uptake of QD-nanoparticles 42 is necessary for the development of a relatively successful imaging tool or drug delivery system.

### Example 3

A mixture of QD-nanoparticles and nanoparticles encapsulating the anti-cancer drug doxorubicin (henceforth referred to as DOX-nanoparticles) were incubated for two hours with CCD-112CoN cell lines.

Incubation was carried out at 37° C. followed by counterstaining of nucleus by 4′-6-Diamidino-2-phenylindole (DAPI). The protocol utilized for this example is the same as that used in Example 2.

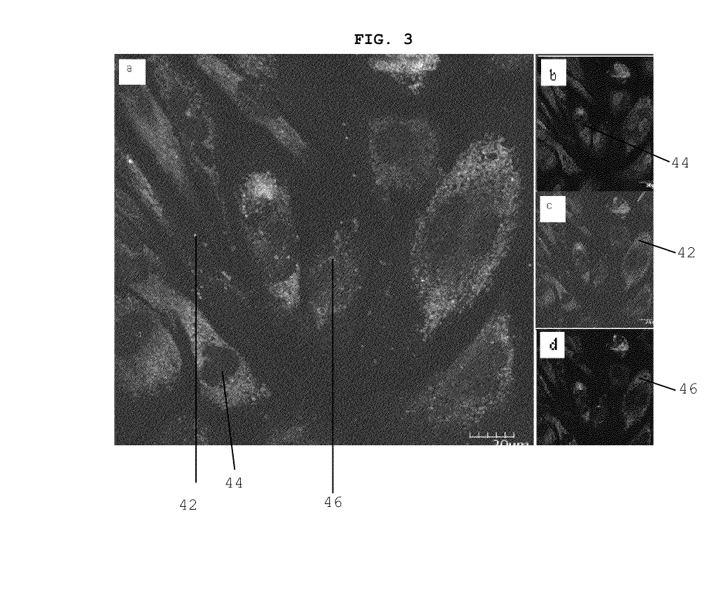

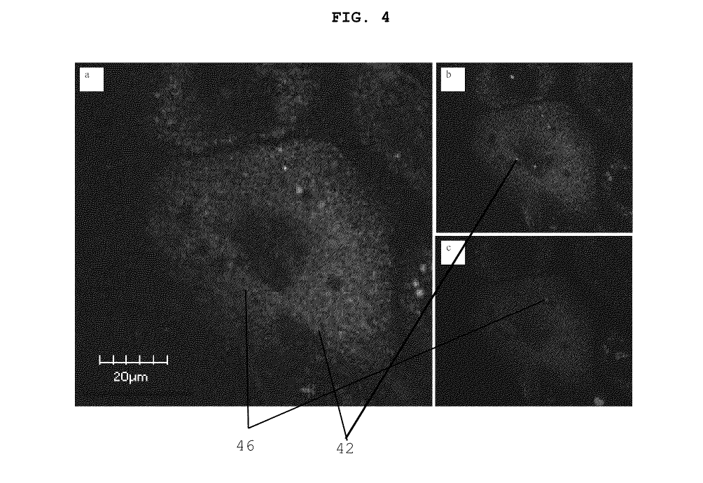

The results are shown in FIG. 3. FIG. 3(a) shows an image of the cells showing co-localization of both QD-nanoparticles 42 and DOX-nanoparticles 46; FIG. 3(b) shows an image showing the stained cell nuclei 44 distinguishing one cell from another; FIG. 3(c) shows an image of the distribution of QD-nanoparticles 42 in cells while FIG. 3(d) shows the distribution of DOX-nanoparticles 46 in the cells.

Referring now to FIG. 4, the three images separately show the confocal fluorescence images of a single cell of the CCD-112CoN cell lines after a two-hour incubation at 37° C. with a mixture of QD-nanoparticles 42 and DOX-nanoparticles 46. FIG. 4(a) shows a magnified view of a single cell showing co-localization of QD-nanoparticles and DOX-nanoparticles 46, visualized by overlaying images. FIG. 4(b) shows an image of the QD-nanoparticles 42 distribution in a single cell while FIG. 4(c) shows an image of the DOX-nanoparticles 46 distribution in a single cell.

The above results indicate that the nanoparticles are effective vectors/carriers for drug delivery into cells. Furthermore, as a result of their ideal optical properties, the extent and efficiency of drug release can be monitored as well. As most drugs are non-fluorescent, the QD-nanoparticles, when also encapsulating a drug or a therapeutic agent in admixture with the QDs, can be used as an imaging-guided chemotherapy system. When QDs are applied as a model of drug or bio-label in a particular drug delivery system formulation or imaging tool development, the QD-nanoparticles can be used as a model system to study the feasibility of any particulate drug delivery system or imaging tool and to study the suitability of the amphiphilic polymer as an encapsulating material.

### Example 4

The experimental procedures in Example 2 and 3 were duplicated for a different cell line, specifically the non-small-cell lung carcinoma (NSCLC) cell line NCI-H1299. Referring now to FIG. 6, FIG. 6 shows the confocal fluorescence image of the QD-nanoparticles 42 uptake in NCI-H1299 cells. The image of the NCI-H1299 cells were taken after a 2 hour incubation period with QD-nanoparticles at 37° C. followed by counterstaining of nucleus 44 by PI.

The above results indicate that the nanoparticles are taken into cells and exhibit fluorescence, thereby proving that they are good fluorophores probes. Furthermore, the above results indicate that the QD-nanoparticles are robust universal tools which can be applied in different cell types.

### Example 5

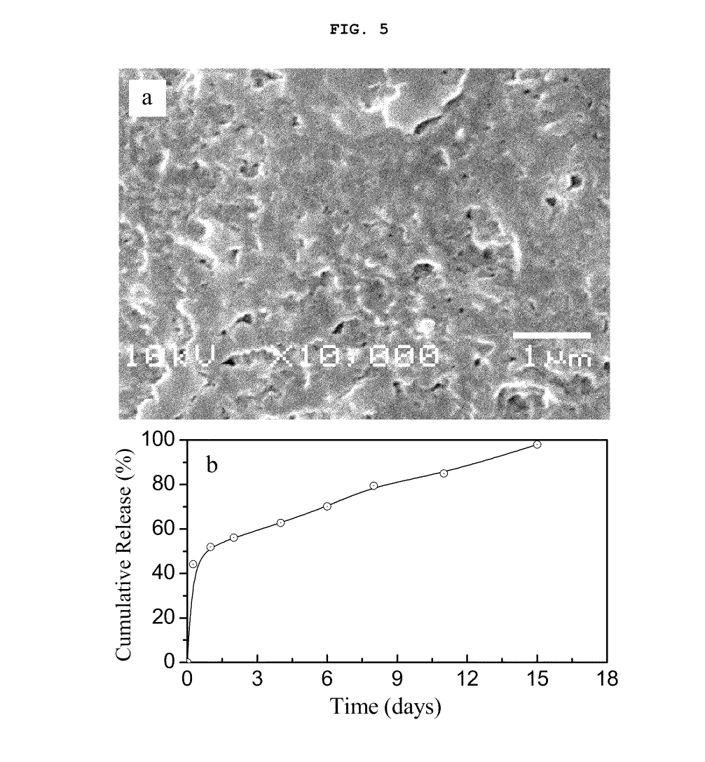

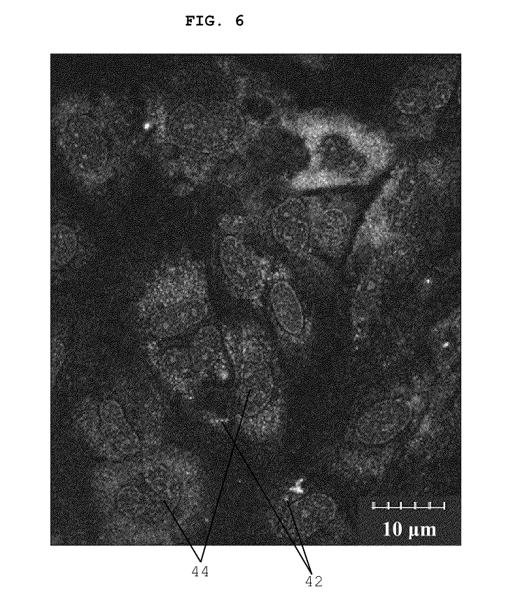

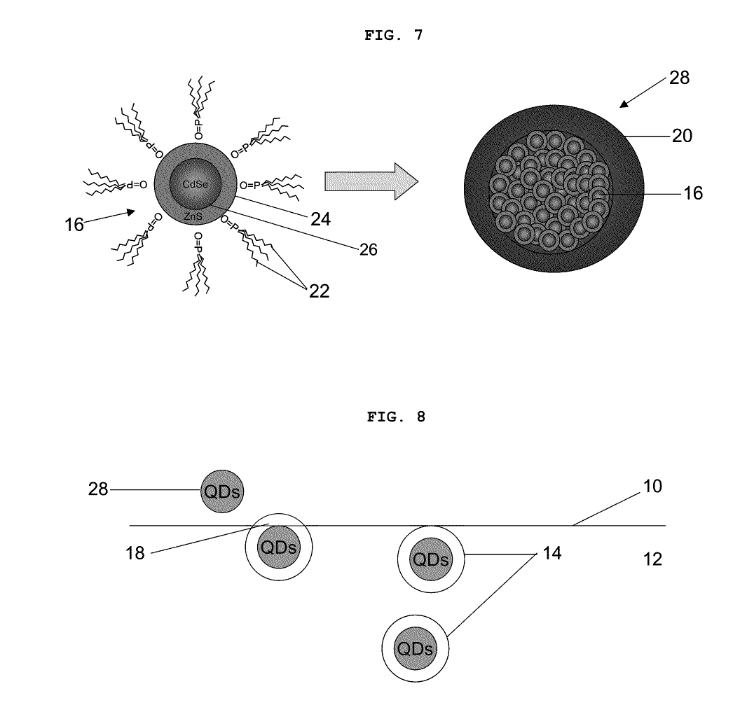

The drug release profile when using the disclosed nanoparticles for drug delivery is investigated in this example. Referring now to FIG. 5(b), the graph shows the DOX release profile representing the cumulative percentage of total DOX released from DOX-nanoparticles in PBS, pH 7.4 at 37° C. over a period of 15 days is charted. The amount of drug released was determined by spectrofluorometric measurement of the released medium and expressed in cumulative released percentage over the original amount of drug encapsulated in DOX-nanoparticles.

As can be seen from the results, at least 50% of the total dosage of DOX was slowly released from day 2 to day 15. This is important with respect to sustaining prolonged therapeutic levels of DOX in a subject. More importantly, these results also ascertain the suitability of the nanoparticles as vectors for drug release and also as means for controlled drug release. The scanning electron micrograph as shown in FIG. 5(a) illustrates the degradation of DOX-nanoparticles after 21 days in a phosphate-buffered saline (PBS) at a pH of 7.4 at and temperature at 37° C. The formulation with the use of an amphiphilic polymer to encapsulate DOX allows for sustained release of DOX in a controlled manner for at least 15 days. The hydrophilic nature of DOX tends to result in a faster release from the particle matrix in the body, leading to the failure of a controlled drug delivery system and the possibility of abrupt overdose above the therapeutical/tolerance level. The prolonged period of steady drug release and the maintenance of drug level in the therapeutic window for an extended time are the major prerequisites for development of controlled drug delivery system. Accordingly, the release profile shows that these DOX-nanoparticles are suitable candidates for a successful drug delivery system.

### APPLICATIONS

The disclosed particle comprising a quantum dot encapsulated by an amphiphilic polymer may be used as an optical marker in vivo. The hydrophilic shell of the amphiphilic polymer may aid in the solubilization of the encapsulated quantum dots in an aqueous medium, while retaining the optical property of the quantum dots. Furthermore, the biocompatibility of the amphiphilic polymer used may aid in promoting the cellular uptake of the disclosed particle into a cell. When the disclosed particle is administered to a mammal, the biocompatible amphiphilic polymer may aid in preventing, or at least reducing, any substantial degradation or scavenging of the disclosed particle by the reticuloendothelial system of the mammal.

The disclosed particle may serve as a model system to study the cell-uptake behaviors of the disclosed particle. By identifying the cell-uptake behaviors of a variety of test particles that comprise the same type of quantum dots encapsulated by a variety of test amphiphilic polymers, the disclosed particles can be used to screen the test amphiphilic polymers as potential candidates for non-fluorescent drug delivery and for controlled release.

The disclosed particle may comprise a therapeutic agent in the place of quantum dot. Accordingly, the disclosed particle may function as a drug delivery vector. The formulated system provides comparable cellular interaction and efficient cellular uptake for both QD-nanoparticles and DOX-nanoparticles. In addition, the DOX-nanoparticles demonstrated sustained release of DOX for an extended period. Thus, the disclosed particle can be used to encapsulate a therapeutic agent or a combination of therapeutic agents to function as effective controlled drug delivery system.

The disclosed particle may further comprise a therapeutic agent in admixture with the quantum dot. Accordingly, the disclosed particle may function as an imaging-guided drug delivery vector. The optical property of the quantum dot may allow for easy visualization or biological imaging of the metabolic pathway or efficacy of the therapeutic agent when the disclosed particle is administered to a mammal.

Advantageously, the symmetric fluorescence emission, photochemical stability and low photodegradation rates of the quantum dots allow for a continuous or long-term real-time monitoring of slow biological processes, tracking of intracellular processes or in cell labeling studies that is not possible with conventional organic fluorophores. In addition, the disclosed particles are useful as means to tag biomolecules when used for bio-imaging applications such as ultrasensitive biological detection and medical diagnostics.

Advantageously, the possibility of tuning the fluorescence emission wavelength of the quantum dots within a broad wavelength range of 400 to 1350 allows for the disclosed particles to be used in bio-imaging applications with greater flexibility over the imaging parameters.

Furthermore, the ability to control or manipulate the size of the quantum dot in order to result in the emission of a preferred colour or emission of a range of colours under varying conditions may allow for a plurality of disclosed particles with varying sizes to be simultaneously excited using a single wavelength to detect different optical activities.

The quantum dots encapsulated by the amphiphilic polymers disclosed herein may not require the use of surface modification or capping agents or additional coating layers. Therefore, the disclosed particles may be easier to make as compared to conventional methods that are used to alter the polarity of the quantum dots.

Furthermore, unlike conventional quantum dots, the disclosed particles may be biocompatible and may be used in vivo.

The disclosed particles may be advantageously used in cancer therapy due to the effective accumulation of polymeric nanoparticles in most types of tumors. Accordingly, the disclosed particles may be used to determine the extent and spread of cancer cells throughout the body during metastasis. In embodiments where a mixture of an anticancer drug and quantum dot is encapsulated by an amphiphilic polymer, the optical property of the quantum dot may aid a medical practitioner in determining the efficacy and therapeutic action of the therapeutic agent on the cancer cells. This may allow for a customized treatment regimen and may enable a medical practitioner to accurately identify cancerous tissues or organs of a mammal suffering from cancer.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A particle comprising a quantum dot encapsulated by an amphiphilic polymer.

2. A particle as claimed in claim 1, wherein the quantum dot is substantially hydrophobic.

3. A particle as claimed in claim 1, wherein the amphiphilic polymer is biocompatible.

4. A particle as claimed in claim 3, wherein the biocompatible polymer is selected from the group consisting of polyesters, poly(orthoester)s, polyanhydrides, poly(amino acid)s, poly(pseudo amino acid)s, and polyphosphazenes.

5. A particle as claimed in claim 4, wherein the polyester is selected from the group consisting of poly(lactic acid)s, poly(glycolic acid)s, copolymers of lactic and glycolic acid, copolymers of lactic and glycolic acid with poly(ethylene glycol), poly(ε-caprolactone)s, poly(3-hydroxybutyrate)s, polybutyrolactones, polypropiolactones, poly(p-dioxanone)s, poly(valerolactone)s, poly(hydrovalerate)s, poly(propylene fumarate)s and derivatives thereof.

6. A particle as claimed in claim 4, wherein said biocompatible polyester is linear or star shaped polyester.

7. A particle as claimed in claim 4, wherein the biocompatible polyester polymer has a molecular weight from about 1,000 Da to about 100,000 Da.

8. A particle as claimed in claim 5, wherein the biocompatible polyester polymer is a poly(lactic-co-glycolic acid).

9. A particle as claimed in claim 8, wherein the ratio of lactic acid and glycolic acid ranges from 1:10 to 10:1.

10. A particle as claimed in claim 3, wherein the biocompatible amphiphilic polymer has a hydrophobic inner core surrounded by a hydrophilic outer skin.

11. A particle as claimed in claim 10, wherein the hydrophilic outer skin comprises hydrophilic functional groups.

12. A particle as claimed in claim 11, wherein the hydrophilic functional groups are selected from the group consisting of hydroxyl groups, carboxyl groups, ether groups, sulfide groups, ester groups, ethoxy groups, phosphonyl groups, phosphinyl groups, sulfonyl groups, sulfinyl groups, sulfonic acid groups, sulfinic acid groups, phosphoric acid groups, phosphorous acid groups, amino groups, amide groups, quaternary ammonium groups, and quaternary phosphonium groups.

13. A particle as claimed in claim 10, wherein the inner core comprises hydrophobic functional groups.

14. A particle as claimed in claim 13, wherein the hydrophobic functional groups are selected from the group consisting of linear or branched alkyl groups, aryl groups, alkenyl groups, alkynyl groups, alkylacrylamide groups, substituted or unsubstituted alkylacrylate groups, and alkylaryl groups.

15. A particle as claimed in claim 1, wherein the size of the particle is in the nanometer range.

16. A particle as claimed in claim 15, wherein the size of the particle is 100 nm to 300 nm in size.

17. A particle as claimed in claim 1, wherein the particle is substantially spherical in shape.

18. A particle as claimed in claim 1, wherein the quantum dot is at least one element selected from the group consisting of Group IIB, Group IVA, Group VA, Group IIIA, Group IIA or Group VIA of the Periodic Table of Elements.

19. A particle as claimed in claim 18, wherein the quantum dot is selected from the group consisting of CdO, CdS, CdSe, CdTe, CdSeTe, CdHgTe, ZnS, ZnSe, ZnTe, ZnO, MgTe, MgS, MgSe, MgO, GaAs, GaP, GaSb, GaN, HgO, HgS, HgSe, HgTe, CaS, CaSe, CaTe, CaO, SrS, SrSe, SrTe, SrO, BaS, BaSe, BaTe, BaO, InAs, InP, InSb, InN, AlAs, AlN, AlP, AlSb, AlS, PbO, PbS, PbSe, PdTe, Ge, Si, ZnO, ZnS, ZnSe, ZnTe and combinations thereof.

20. A particle as claimed in claim 18, wherein the quantum dot is of a core-shell structure.

21. A particle as claimed in claim 1, wherein the quantum dot is an inner core of CdSe that is encapsulated by poly(lactic-co-glycolic acid).

22. A particle as claimed in claim 1, further comprising a therapeutic agent encapsulated by said amphiphilic polymer.

23. Use of a particle comprising a quantum dot encapsulated by an amphiphilic polymer as a fluorescent probe.

24. Use of a particle comprising a quantum dot and a therapeutic agent encapsulated by an amphiphilic polymer for controlled release of said therapeutic agent in a patient, wherein said quantum dot is capable of being optically detected in vivo during said release.

25. A method of preparing an encapsulated quantum dot comprising the step of introducing an aqueous solvent to a quantum dot in mixture with an amphiphilic polymer dissolved in an organic solvent to thereby precipitate said polymer and encapsulate said quantum dot.
